(19) United States
(12) Patent Application Publication
(10) Pub. No.: US 2024/0207048 A1
(43) Pub. Date: Jun. 27, 2024

(54) DELIVERY SYSTEM FOR A BALLOON-EXPANDABLE TRANSCATHETER VALVE IMPLANTATION

(71) Applicant: Braile Biomedica Industria Comercio e Representaco, São Paulo (BR)

(72) Inventors: Rafael Braile Cunha, São Paulo (BR); Jose Honorio De Almeida Palma da Fonseca, São Paulo (BR); Vinicius Ladeia Semenzim, São Paulo (BR); Fabio Da Silva, São Paulo (BR)

(21) Appl. No.: 18/229,408

(22) Filed: Aug. 2, 2023

Related U.S. Application Data
(60) Provisional application No. 63/394,363, filed on Aug. 2, 2022.

(51) Int. Cl. A61F 2/24

(52) U.S. Cl. CPC A61F 2/2433

(57) ABSTRACT

A delivery system for a balloon-expandable transcatheter valve implantation is disclosed. The delivery system includes an anti-inflation lock mechanism to prevent improper inflation of a balloon. A sheath includes a flared portion positioned at a tip to partially sheathe a transcatheter valve during blood vessel navigation. The sheath houses the balloon during an implantation procedure. A reinforced balloon catheter shaft prevents the balloon from kinking within a blood vessel. A self-centering system is configured to center the balloon within the aortic annulus before the implantation procedure.

### TECHNICAL FIELD

The embodiments generally relate to catheters and more specifically to delivery systems for balloon-expandable transcatheter valve implantation.

### BACKGROUND

Endovascular delivery catheters are used to implant prosthetic devices, such as a prosthetic valve, at locations inside the body that are not readily accessible by surgery or where access without surgery is desirable. The usefulness of delivery catheters is largely limited by the ability of the catheter to successfully navigate through small vessels and around tight bends in the vasculature, such as around the aortic arch.

Known delivery apparatuses include a balloon catheter having an inflatable balloon that mounts a prosthetic valve in a crimped state and a retractable cover that extends over the valve to protect the interior walls of the vasculature as the valve is advanced to the implantation site. Various techniques have been employed to adjust the curvature of a section of the delivery apparatus to help “steer” the valve through bends in the vasculature. The balloon catheter may also include a tapered tip portion mounted distal to the balloon to facilitate tracking through the vasculature. The tip portion, however, increases the length of the relatively stiff, non-steerable section of the apparatus. Unfortunately, due to the relatively long stiff section, successful delivery of a prosthetic valve through tortuous vasculature, such as required for retrograde delivery of a prosthetic aortic heart valve, has proven to be difficult.

A known technique for adjusting the curvature of a delivery apparatus employs a pull wire having a distal end fixedly secured to the steerable section and a proximal end operatively connected to a rotatable adjustment knob located outside the body. Rotation of the adjustment applies a pulling force on the pull wire, which in turn causes the steerable section to bend. The rotation of the adjustment knob produces less than 1:1 movement of the pull wire; that is, rotation of the knob does not produce equal movement of the steerable section. To facilitate steering, it would be desirable to provide an adjustment mechanism that can produce substantially 1:1 movement of the steerable section.

It is also known to use an introducer sheath for safely introducing a delivery apparatus into the patient's vasculature (e.g., the femoral artery). An introducer sheath has an elongated sleeve that is inserted into the vasculature and a seal housing that contains one or more sealing valves that allow a delivery apparatus to be placed in fluid communication with the vasculature with minimal blood loss. A conventional introducer sheath typically requires a tubular loader to be inserted through the seals in the sheath housing to provide an unobstructed path through the seal housing for a valve mounted on a balloon catheter. A conventional loader extends from the proximal end of the introducer sheath, and therefore decreases the available working length of the delivery apparatus that can be inserted through the sheath and into the body.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provide a delivery system for a balloon-expandable transcatheter valve implantation. The delivery system includes an anti-inflation lock mechanism to prevent the operator to perform the inflation, or the expansion, of the balloon when the system is not ready (balloon totally free): The main advantage of this new feature is to prevent balloon inflations during the medical procedure when the balloon is not fully free of the sheath. During the navigation of the system, the balloon is positioned inside the sheath to support the transcatheter valve inside blood vessels until the site of implantation (navigation stage of the system). When the balloon is inside the sheath, by its instructions of use, its inflation is not performed. When it occurs, the inflation is partial and some complications may happen, such as valve migration, loss of ideal positioning, and system damages/failures. Thus, an advantage is to avoid complications caused by human operation (mostly caused by distractions, or lack of training on the usage), increasing the safety of the device.

The delivery system includes a sheath with a flared portion in the tip to partially sheathe the transcatheter valve during the blood vessel navigation. This has the advantage of supporting the valve during its navigation with the delivery system, and also protects the valve stent against some contact with the walls of the blood vessels. When the navigation occurs, the transcatheter valve (crimped on the balloon) is supported by the sheath to stabilize the valve in its position. This feature also creates strong pushability across the calcified annulus of the system.

A reinforced balloon catheter shaft is provided with the delivery system to prevent the balloon kinking of the system into blood vessel tortuosity. This feature is applied to increase the pushability of the balloon when the transcatheter valve is loaded on it. It reinforces the balloon shaft to avoid “balloon bends” at the moment to cross the calcified aortic annulus.

A self-centering system is provided with the delivery system and is boarded in the same delivery system to self-centralize the balloon inside the aortic annulus before the implantation. This feature has the advantage to create a mechanism that pushes the adjacent structures (calcified tissues), and it is an auxiliary device to find the ideal coaxial alignment in the aortic annulus.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIGS. 1A-B illustrate a side elevation view of the anti-inflation lock of the delivery system, according to some embodiments;

FIG. 2 illustrates a partial side elevation view of the semi-sheathing configuration used during the implementation stage, according to some embodiments;

FIG. 3 illustrates a partial side elevation view of the reinforced balloon shaft, according to some embodiments; and

FIG. 4 illustrates a perspective view of the self-centering mechanism, according to some embodiments.

### DETAILED DESCRIPTION

The specific details of the single embodiment or variety of embodiments described herein are to the described apparatus. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the apparatus. Accordingly, the apparatus components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The specific details of the single embodiment or variety of embodiments described herein are set forth in this application. Any specific details of the embodiments are used for demonstration purposes only, and no unnecessary limitation or inferences are to be understood therefrom. Furthermore, as used herein, relational terms, such as “first” and “second,” “top” and “bottom,” and the like, may be used to distinguish one entity or element from another entity or element without necessarily requiring or implying any physical or logical relationship, or order between such entities or elements.

In general, the embodiments described herein relate to a delivery system for a balloon-expandable transcatheter valve implantation. The delivery system prevents the operator from performing the inflation, or the expansion, of the balloon when the system is not ready (i.e., balloon totally free). The main advantage of this new feature is to prevent balloon inflations during the medical procedure when the balloon is not fully free of the sheath. During the navigation of the system, the balloon is positioned inside the sheath to support the transcatheter valve inside blood vessels until the site of implantation (navigation stage of the system). When the balloon is inside the sheath, by its instructions of use, its inflation must not be performed. When it occurs, the inflation is partial and some complications may happen, such as valve migration, loss of ideal positioning, and system damages/failures.

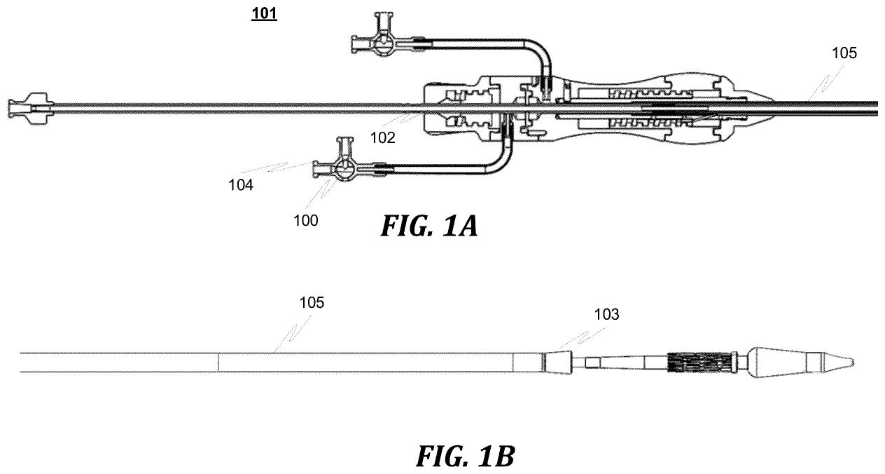

FIG. 1A-B illustrate a side elevation view of the anti-inflation lock 100 of the delivery system 101. The anti-inflation lock mechanism prevents the operator from inflating, or expanding, the balloon 103 (see FIG. 1B) when the system is not ready (i.e., when the balloon is not yet fully free). The navigation stage 102 of the system 101 may control the balloon movement. The anti-inflation lock is a safety device inserted on the balloon line/luer 104 that blocks the passage of fluid and does not allow balloon inflations when the system is not at the ready-inflation stage or the balloon-free stage. The non-ready stage may be defined as when the balloon is inside the sheath 105.

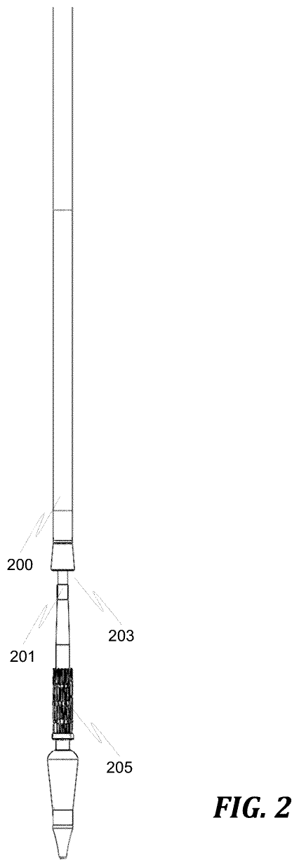

FIG. 2 illustrates a partial side elevation view of the semi-sheathing configuration used during the implementation stage. The sheath 200 includes a flared portion 201 in the tip 203 to partially sheathe the transcatheter valve 205 during the blood vessels navigation portion of the procedure (i.e., during the implementation stage). The sheath is steerable including the balloon catheter therein. The steerable sheath acts as a guide of the tip of the balloon as well as a shaft to support the transcatheter valve 205. A handle is connected to the balloon catheter and the steerable sheath 200 to allow the operator to flex and move (forward and backward) the tip 203 containing the balloon. An extension of the balloon catheter crosses inside the handle and ends in a guidewire port.

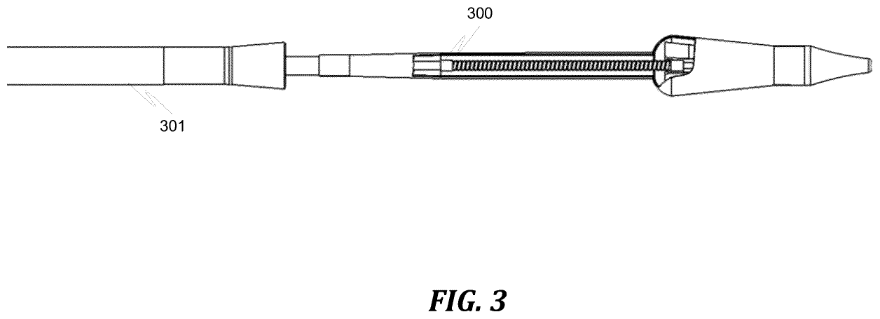

FIG. 3 illustrates a partial side elevation view of the reinforced balloon shaft 300. The reinforced balloon catheter shaft 300 prevents the balloon from kinking or other damaging actions of the delivery system 301 into the blood vessel's tortuosity.

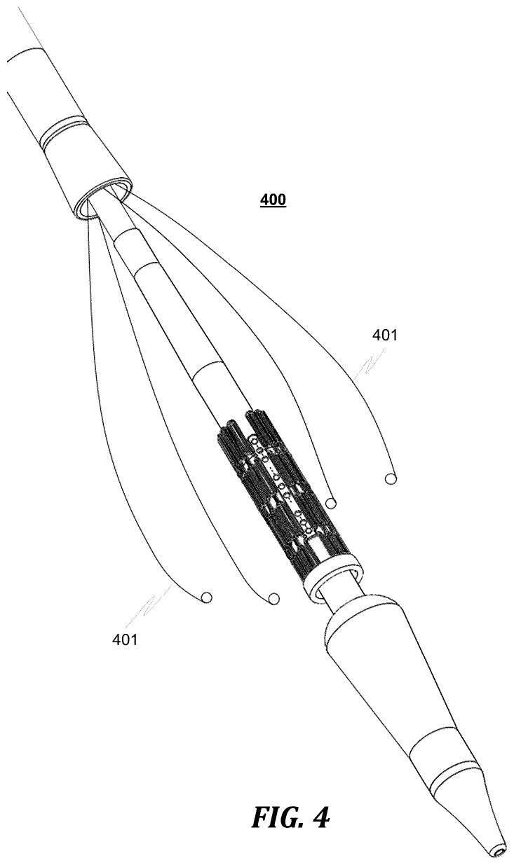

FIG. 4 illustrates a perspective view of the self-centering mechanism 400. The self-centering mechanism 400 is housed in the delivery system to self-centralize the balloon inside the aortic annulus before the implantation procedure. The self-centering mechanism 400 is composed of wires 401 inside the steerable sheath that is triggered in the handle when the valve is positioned in the aortic annulus. The wires 401 are pushed outside the sheath to create a cage that accommodates the adjacent structures (annulus) and forces the balloon to be in the center of the annulus and allows central balloon expansion.

The delivery system for the balloon-expandable transcatheter valve implantation may be in combination with its respective valves. For balloon-expandable transcatheter valves, it is necessary to have a balloon on it due to its functionality of expansion. The inflation of the balloon is performed by the medical operator (generally a physician), who connects the balloon hub/port to a syringe with fluid. This fluid is inserted into the balloon line/luer to inflate it and expand the valve.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A delivery system for a balloon-expandable transcatheter valve implantation, comprising:
an anti-inflation lock mechanism to prevent improper inflation of a balloon;
a sheath comprising a flared portion positioned at a tip of the sheath to partially sheathe a transcatheter valve during blood vessel navigation; and
a reinforced balloon catheter shaft configured to prevent the balloon from kinking within a blood vessel.

2. The system of claim 1, further comprising a self-centering mechanism configured to center the balloon within an aortic annulus before the implantation procedure.

3. The system of claim 2, wherein the self-centering mechanism comprises a plurality of wires configured to be pushed outside the sheath to create a cage that accommodates the annulus and forces the balloon to be in a center of the annulus to allow for a central balloon expansion.

4. The system of claim 1, wherein the sheath is configured to house the balloon during an implantation procedure.

5. The system of claim 1, wherein the anti-inflation lock mechanism is configured to prevent an operator from inflating or expanding the balloon when the balloon is not completely freed up.

6. The system of claim 1, wherein the anti-inflation lock is configured to be positioned on a balloon luer that blocks passage of fluid to prevent the balloon from inflations.

7. The system of claim 1, wherein the sheath is steerable and configured to house a balloon catheter therein.

8. The system of claim 7, wherein the steerable sheath is configured to guide a tip of the balloon and support the transcatheter valve.

9. The system of claim 7, further comprising a handle is connected to the balloon catheter and the steerable sheath to allow an operator to flex and move tip of the balloon.

10. The system of claim 9, wherein an extension of the balloon catheter crosses inside the handle and ends in a guidewire port.

11. The system of claim 1, further comprising a self-centering mechanism comprising a plurality of wires placed inside a steerable sheath configured to be triggered in a handle when transcatheter valve is positioned in an aortic annulus.
